SEQUENCE LISTING (3 sequences)

SEQ ID NO: 1 - DNA, 20 bp, Homo sapiens
tgtgccaacgagaccaagac

SEQ ID NO: 2 - DNA, 22 bp, Homo sapiens
cgtgctcaaaggagtcatcaaa

SEQ ID NO: 3 - DNA, 15 bp, Homo sapiens
aggcaaggtgctcat

(19) United States
(12) Patent Application Publication
(10) Pub. No.: US 2008/0124718 A1
(43) Pub. Date: May 29, 2008

(54) METHODS OF DIAGNOSING, PREDICTING THERAPEUTIC EFFICACY AND SCREENING FOR NEW THERAPEUTIC AGENTS FOR LEUKEMIA

(75) Inventors: Eva Otahalova, Kobyli (CZ); Michael Lebowitz, Baltimore, MD (US); Hossein A. Ghanbari, Potomac, MD (US)

Correspondence Address: PANACEA PHARMACEUTICALS, INC., 207 PERRY PARKWAY, SUITE 2, GAITHERSBURG, MD, 20877, US

(73) Assignee: Panacea Pharmaceuticals, Inc., Gaithersburg, MD (US)

(21) Appl. No.: 11/668,421

(22) Filed: Jan. 29, 2007

Related U.S. Application Data
(60) Provisional application No. 60/843,680, filed on Sep. 11, 2006.
(60) Provisional application No. 60/762,590, filed on Jan. 27, 2006.

(51) Int. Cl. C12Q 1/68; C12Q 1/02

(52) U.S. Cl. 435/6; 435/29

(57) ABSTRACT

The invention discloses methods for leukemia diagnosis and determining the effectiveness of certain therapies. The methods of the present invention also encompass a way to predict a subject's responsiveness to therapeutic interventions for leukemia, as well as to monitor relapse during treatment due to therapeutic resistance. Further, the methods disclosed can be used to screen for effective therapeutic agents or regimens, either generally or in a specific patient. The invention also provides a unique diagnostic tool for leukemia.

The application claims priority to provisional applications U.S. Ser. Nos. 60/762,590, filed Jan. 27, 2006 and 60/843,680, filed Sep. 11, 2006, the contents of which are incorporated by reference herein in their entireties.

### FIELD OF THE INVENTION

The invention is in the field of leukemia diagnosis and treatment, generally, and more specifically is related to determining the effectiveness of certain therapies. The methods of the present invention encompass a simple, yet elegant, way to predict a subject's responsiveness to therapeutic interventions for leukemia, as well as to monitor relapse during treatment due to therapeutic resistance. Moreover, the methods can be used to screen for effective therapeutic agents or regimens, either generally or in a specific patient. Still further, a unique diagnostic tool for leukemia is established by the discoveries and techniques disclosed.

### BACKGROUND OF THE INVENTION

Leukemia is not the result of a single, well-defined cause, but rather can be viewed as several diseases, each caused by different aberrations in genes and biochemical pathways, which ultimately result in apparently similar pathologic phenotypes. The identification of genes that are differentially expressed in leukemia cells relative to normal cells of the same tissue type provides the basis for diagnostic tools, facilitates drug discovery by providing for unique targets of the disease, and further serves to predict the therapeutic efficacy of known drugs in individual patients.

The enzyme, aspartyl (asparaginyl) β-hydroxylase (aka “AAH” or “ASP”), has been shown to be overexpressed, in comparison to normal controls, in all malignant tumors of endodermal origin and in at least 95% of CNS tumors studied to date. Malignant neoplasms detected in this manner include those derived from endodermal tissue, e.g., colon cancer, breast cancer, pancreatic cancer, liver cancer, and cancer of the bile ducts. Neoplasms of the central nervous system (CNS) such as primary malignant CNS neoplasms of both neuronal and glial cell origin and metastatic CNS neoplasms are also detected. Patient derived tissue samples, e.g., biopsies of solid tumors, as well as bodily fluids such as a CNS-derived bodily fluid, blood, serum, urine, saliva, sputum, lung effusion, and ascites fluid, are contacted with an HAAH-specific antibody. See further, Wands et al., U.S. Pat. Nos. 6,797,696; 6,783,758; 6,812,206; 6,815,415; 6,835,370; and 7,094,556, each of which is hereby incorporated by reference in its entirety.

### SUMMARY OF THE INVENTION

Now it has been found that human AAH (also referred to herein as “HAAH” and also “ASPH”) is overexpressed in leukemic cells, which are not solid malignancies. This discovery has many implications. In one aspect of the present invention, AAH provides an excellent marker for drug discovery, as well as a marker for efficacy of or sensitivity to various therapeutic interventions in leukemia, especially since it has been found to have decreased expression even in instances in which it is not the target of the therapeutic agent (e.g. Gleevec®). In other words, down-regulation of AAH is considered a universal marker of treatment success in leukemia, whether this enzyme is the therapeutic target or not.

A major challenge of treatment of leukemia is the selection of therapeutic agents and regimens that maximize efficacy and minimize toxicity, even for an individual subject. A related challenge lies in the attempt to provide accurate diagnostic, prognostic and predictive information for leukemia. There clearly exists a need for improved methods and reagents for accomplishing these goals.

Now that the present inventors have discovered the marker for such determinations (AAH), clinical evaluations can be performed that will allow the identification of those patients having different prognoses and/or responses to a given therapy, or in the identification of relapse of the illness during therapy. Clearly, this prognostic tool will allow more rational choices of the best course and drug to be used in therapeutic interventions, and direct patients to the most appropriate treatments.

In another aspect of the present invention, AAH expression can be used to screen for potentially effective therapies against leukemia. A method of screening for potential therapeutic agents is provided by measuring the expression of AAH in samples containing leukemia cells as compared to corresponding samples containing normal lymphocytes.

In human leukemia patients, HAAH is vastly overexpressed in leukemia cells, as evidenced from peripheral blood lymphocytes of patients with Chronic Myelogenous Leukemia (CML) and Acute Myelogenous Leukemia (AML), as compared to normal controls (i.e., on the order of about 80 times higher). This gene overexpression represents a valuable tool for diagnosis, for assessing whether a given patient is responding or will respond to treatment with a particular agent, and for screening candidate drugs in a broader sense, because it has now been found that a decrease in HAAH expression is indicative of responsiveness to drug treatment.

### BRIEF DESCRIPTION OF THE DRAWINGS

FIG. 1 is a graph of HAAH gene expression in leukemia patients vs. healthy donors.

FIG. 2 is a graph showing changes in HAAH gene expression in response to Gleevec® treatment.

FIGS. 3 and 4 are graph showing that Ki67 and BCR-ABL gene expression, respectively, are not predictive of responsiveness to Gleevec®.

FIG. 5 is a graph depicting HAAH gene expression in lymphocytes of normal, AML untreated and AML treated individuals.

FIG. 6 is a graph showing HAAH gene expression comparing Gleevec® responders and non-responders.

### DETAILED DESCRIPTION OF THE INVENTION

Chronic Myelogenous Leukemia (CML) is a malignant clonal disorder of hematopoietic stem cells resulting in increases in myeloid and erythroid cells as well as platelets in peripheral blood, and marked myeloid hyperplasia in the bone marrow. CML accounts for 15% of leukemias in adults. The median age at presentation is 53 years, but all age groups, including children, are affected.

The molecular hallmark of CML is the Philadelphia (Ph) chromosome, a shortened version of chromosome 22 that is the result of a translocation between chromosomes 9 and 22 and is found in 95% of CML patients. The Ph translocation results in the recombination of segments from the BCR and ABL genes creating a hybrid BCR-ABL gene. This hybrid gene is transcribed and translated to produce the BCR-ABL fusion protein, an unregulated tyrosine kinase (TK) that alters cellular growth and function.

Currently, CML is confirmed by the presence of an elevated WBC count, splenomegaly, thrombocytosis, and identification of the BCR/ABL translocation. Cytogenetic karyotyping, which is the standard diagnostic test for CML, not only can detect the Ph chromosome in most CML patients, but can also detect the presence of other clinically significant chromosomal abnormalities.

Imatinib mesylate (Gleevec®) has emerged as the first-line therapy for CML. Imatinib is a TK inhibitor with high specificity for the BCR-ABL protein. While imatinib induces complete cytogenetic remission (CCR) in the majority of patients, a significant number of individuals do not respond and would benefit from alternative therapies earlier in the course of disease. Although the hybrid BCR-ABL gene is the molecular hallmark of CML, mutations to the ABL portion of the gene do not reliably predict response to imatinib therapy.

Imatinib is molecularly-specific oral anticancer agent that selectively inhibits several protein tyrosine kinases central to the pathogenesis of human cancer. It has demonstrated remarkable clinical efficacy in patients with Chronic Myeloid Leukemia (CML), as well as other diseases such as malignant gastrointestinal stromal tumors (GIST). Imatinib was first made available to patients with CML in May of 2001. Imatinib is indicated for the treatment of newly diagnosed adult patients with Philadelphia chromosome-positive (Ph+) chronic myeloid leukemia (CML) in chronic phase. Imatinib is also indicated for the treatment of patients with Ph+CML in blast crisis, accelerated phase, or in chronic phase after failure of interferon-alpha therapy. Imatinib is also indicated for the treatment of pediatric patients with Ph+chronic phase CML whose disease has recurred after stem cell transplant or who are resistant to interferon-alpha therapy. In late 2006, the US Food & Drug Administration expanded the approval of Gleevec® to include treatment as a single agent in dermatofibrosarcoma protruberans (DFSP), myelodysplastic/myeloproliferative diseases (MDS/MPD), aggressive systemic mastocytosis (ASM), hypereosinophilic syndrome/chronic eosinophilic leukemia (HES/CEL), and relapsed/refractory Philadelphia chromosome positive acute lymphocytic leukemia.

In vivo, it inhibits tumor growth of BCR-ABL transfected murine myeloid cells as well as BCR-ABL positive leukemia lines derived from CML patients in blast crisis. Imatinib is also an inhibitor of the receptor tyrosine kinases for platelet-derived growth factor (PDGF) and stem cell factor (SCF), c-kit, and inhibits PDGF- and SCF-mediated cellular events. In vitro, imatinib inhibits proliferation and induces apoptosis in gastrointestinal stromal tumor (GIST) cells, which express an activating c-kit mutation.

Treatment with imatinib is generally well tolerated, and the risk for severe adverse effects is low. Adverse effects most commonly include mild-to-moderate edema, nausea and vomiting, diarrhea, muscle cramps, and cutaneous reactions. Hepatic transaminase level elevations and myelosuppression occur less frequently and resolve with interruption of imatinib therapy. In general, the incidence and severity of adverse effects tend to correlate with imatinib dose and, in chronic myeloid leukemia patients, the phase of disease; but, patient age and other factors are also associated with some types of reactions. With prompt and appropriate intervention, adverse effects in imatinib-treated patients have proven to be manageable across the spectrum of severity, and they seldom require permanent cessation of therapy. Dose reduction is not usually necessary, and reduction to subtherapeutic levels is not recommended.

The majority of patients who received Gleevec® in clinical trials did experience adverse effects. Most adverse effects were mild or moderate. The most common adverse effects were fluid retention, nausea, muscle cramps, diarrhea, vomiting, muscle and bone pain, fatigue, rash, and abdominal pain. Serious and severe adverse effects such as liver problems, water retention in different parts of the body including the heart, muscle or bone pain, skin blistering, and low levels of certain blood cells have also been reported in some patients. Gleevec® was discontinued due to adverse effects in 4 percent of patients in the chronic phase of CML, 5 percent in accelerated phase, 5 percent in blast crisis, and 8 percent in GIST. Recently, reports of cardiotoxicity following initiation of treatment with Gleevec® have been reported (Kerkela, et. al., Cardiotoxicity of the cancer therapeutic agent imatinib mesylate. Nature Medicine, 12; 8:908-916. 2006). Clinical trials of involving Gleevec® have reported a relatively high incidence of peripheral edema (63-66%), some of which have been classified as severe (4-5%) In addition, dyspnea has been reported in 12-16% of treated individuals and has been classified as severe in 4-5%. These symptoms have been interpreted to represent left ventricular dysfunction and even frank congestive heart failure.

Gleevec® has become recognized as the most effective non-transplant treatment available for patients with CML. However, it is an expensive drug. Costs of the drug range from $30,000 to $40,000 per year (USA Today, Cost of cancer drugs crushes all but hope. Jul. 11, 2006). The costs per quality adjusted life year is approximately $40,000 more than conventional therapy for patients treated in the accelerated phase and almost $60,000 more for patients treated in blast crisis (Gordois, et. al., Cost-utility analysis of imatinib mesilate for the treatment of advanced stage chronic myelogenous leukemia. British Journal of Cancer 2003; 89:634-640).

Clearly, identifying patients with a low likelihood of responding to imatinib may avoid potentially serious adverse effects, hasten the initiation of potentially more beneficial treatment, and save considerable costs.

Human Aspartyl (asparaginyl) β-Hydroxylase (HAAH) is a cancer biomarker. HAAH is a β-ketoglutarate dependent dioxygenase that catalyzes the hydroxylation of the β-carbon in aspartyl and/or asparaginyl residues found in EGF-like domains of substrate proteins. HAAH is a highly specific biomarker for cancer. Increased HAAH expression has previously been detected at the protein and mRNA levels specifically in tumor cells in more than 20 different solid cancer types including lung, liver, colon, pancreas, prostate, ovary, bile duct, and breast.

The HAAH assay is an in vitro diagnostic device for the quantitative measurement of HAAH in human serum. Cellular transformation to an aggressively invasive phenotype results in significant up-regulation of HAAH transcription and translocation of the enzyme to the cellular surface. The overexpression of HAAH has been detected by immunohistochemical staining in more than 20 different cancer types and in >98% of all tumor samples tested to date (n>1000); but not in significant amounts in adjacent non-affected tissue (n>500).

Preliminary results of the HAAH assay have determined a specificity of 97% (n=230) and a sensitivity of 94% (n=85) for a series of cancers including breast, ovarian, prostate, colon, esophageal, bladder and kidney.

The present inventors have now found that HAAH expression is increased in leukemia as well. Moreover, the present inventors have determined that expression of the gene encoding human aspartyl (asparaginyl) β-hydroxylase (HAAH) significantly decreases when the leukocytes of patients with Chronic Myelogenous Leukemia (CML) are cultured in the presence of imatinib. This decrease in HAAH gene expression level correlates with drug response. Patient non-responders to imatinib treatment do NOT show a decrease in HAAH expression in the assay.

Further, the expression of HAAH can be used to diagnose, monitor and determine drug susceptibility in subjects with AML. See further, Examples 2 and 3.

Diagnosis of Leukemia

One aspect of the present invention relates to a method of detecting leukemic cells in a sample, including as a confirmatory diagnosis of the disease, for disease progression, relapse, or remission, and for testing cells to be used for bone marrow or peripheral blood stem cell transplantation.

The expression level of the AAH gene, as correlated to AAH mRNA, can be determined by the well-known reverse transcribed polymerase chain reaction, or “RT-PCR”, as described in Kawasaki, E. S. et al., Amplification of RNA, in PCR Protocols, A Guide to Methods and Applications, Academic Press, Inc., San Diego, 21-27 (1991).

In order to ensure a positive outcome in the case of bone marrow or stem cell transplantation, it is essential to confirm that the donor cells to be transplanted are free of leukemic cells. This can be accomplished by using the methods described herein.

Thus, an object of the present invention is to provide methods for determining (or quantitating) the presence of leukemic cells, which is based on the discovery that the AAH gene expression/protein level is a valuable clinical marker correlated with leukemia.

Such methods comprise determining if AAH is overexpressed in a test sample as compared to a normal sample. AAH is overexpressed in human leukemic cells, as shown in CML and AML examples of this application. An increased presence of AAH gene product in a patient-derived blood sample is carried out using any standard methodology that measures levels (as compared to known normal controls) of a certain protein, e.g., by Western blot assays or a quantitative assay such as ELISA. For example, a standard competitive ELISA format using an HAAH-specific antibody is used to quantify human patient HAAH (i.e., human AAH) levels. Alternatively, a sandwich ELISA using a first antibody as the capture antibody and a second HAAH-specific antibody as a detection antibody is used.

As the result of intensive research, the inventors of the present invention discovered for the first time that the AAH gene, which is known as a clinical marker of solid tumors, also shows a significant level of expression in leukemias, which are unrelated to solid tumors. Accordingly, it has now been found that the AAH gene (and its expressed protein) can be effectively utilized in the detection of leukemia in a subject.

Methods of detecting AAH include contacting peripheral blood lymphocytes (PBLs) in a sample of blood with an AAH-specific antibody bound to solid matrix, e.g., microtiter plate, bead, dipstick. For example, the solid matrix is dipped into a patient-derived blood sample (or component thereof containing lymphocytes), washed, and the solid matrix is contacted with a reagent to detect the presence of immune complexes present on the solid matrix.

The nature of the solid surface may vary depending upon the assay format. For assays carried out in microtiter wells, the solid surface is the wall of the well or cup. For assays using beads, the solid surface is the surface of the bead. In assays using a dipstick (i.e., a solid body made from a porous or fibrous material such as fabric or paper) the surface is the surface of the material from which the dipstick is made. Examples of useful solid supports include nitrocellulose (e.g., in membrane or microtiter well form), polyvinyl chloride (e.g., in sheets or microtiter wells), polystyrene latex (e.g., in beads or microtiter plates), polyvinylidine fluoride (known as IMMULON®), diazotized paper, nylon membranes, activated beads, and Protein A beads. The solid support containing the anti-AAH antibody is typically washed after contacting it with the test sample, and prior to detection of bound immune complexes. Incubation of the antibody with the test sample is followed by detection of immune complexes by a detectable label. For example, the label is enzymatic, fluorescent, chemiluminescent, radioactive, or a dye. Assays which amplify the signals from the immune complex are also known in the art, e.g., assays which utilize biotin and avidin.

Anti-AAH antibodies useful for AAH detection are, for example, those disclosed in the patents of Wands et al., supra (which are produced by hybridomas that have been deposited with the ATCC), including fragments and derivatives (e.g., labeled) thereof.

An AAH-detection reagent, e.g., one or more anti-AAH antibodies (or immunologically reactive fragments or derivatives thereof), may be commercially distributed alone, or packaged in the form of a kit with other items, such as control formulations (positive and/or negative), and a detectable label. The assay may be in the form of a standard two-antibody sandwich assay format known in the art. As a preferred embodiment for an assay of protein levels is a FACS assay, whereby antibody is allowed to react with the cells, such that the anti-AAH antibody will binds to any AAH on the cell surface. Levels can then be determined by fluorescence activated cell sorting protocols.

While methods for directly diagnosing leukemia are well practiced in the art, the diagnostic method of the present invention can be an adjunct to initial diagnosis, or may be used quantitatively to assess a clinical stage of the disease. As such, as the illness progresses, increasing amounts of AAH expression (e.g., mRNA or protein levels) in periodic test samples of a patient over time would be indicative of a worsening disease state; conversely, decreasing amounts of AAH expression would indicate improvement of the patient's condition.

Screening for Therapeutic Agents

In another aspect, the present invention provides a method for screening potential therapeutic agents (or combinations of agents) for leukemia. Essentially, the method comprises contacting a candidate agent with a known sample containing leukemic cells, such as from a subject with AML or CML, in vitro for a predetermined time, and then measuring AAH protein (such as by an FACS assay, which measures AAH on the cell surfaces) or gene expression (such as through an RT-PCR-type assay, which measures mRNA as an indication of gene expression), and comparing that level to a corresponding protein or gene expression level of AAH of a control leukemic cell sample in the absence of the candidate drug. (Alternatively, gene expression/protein levels can be determined in a single sample of leukemic cells both before and after exposure to the candidate drug to assess the drug's effectiveness.)

The therapeutic agent being evaluated is not limited to any particular substance or class, and may be, for instance, a small molecule, a peptide, an antibody, or an antisense polynucleotide. Interestingly, the candidate being evaluated does not necessarily have to interact with AAH directly; the successful candidate need only have an indirect negative modulation (i.e., inhibitory effect) on AAH expression or activity. Such candidate drugs (or test compounds) may be obtained from any available source, including (combinatorial) libraries produced from biological, natural and/or synthetic compounds.

The basic principle of the screening assay is to identify compounds that inhibit AAH expression, AAH protein levels (or indirectly, AAH activity) in diseased (leukemic) cells. The level of AAH expression through detection of AAH nucleic acid may be determined in any known manner, such as qRT-PCR. The amount of AAH protein in the samples can also be measured by any available method that measures levels of a specific protein in a sample, such as immunological assays.

AAH catalyzes the posttranslational modification of the β carbon of aspartyl and asparaginyl residues of EGF-like polypeptide domains. An assay to identify compounds which directly inhibit this hydroxylase activity, or indirectly show a decrease in this activity due to decreased amounts of AAH in a test sample, is carried out by comparing the level of hydroxylation in an enzymatic reaction in which the candidate compound is present compared to a parallel reaction in the absence of the compound (or a predetermined control value). Standard in vitro hydroxylase assays are known in the art, e.g., Lavaissiere et al., 1996, J. Clin. Invest. 98:1313 1323; Jia et al., 1992, J. Biol. Chem. 267:14322 14327; Wang et al., 1991, J. Biol. Chem. 266:14004 14010; or Gronke et al., 1990, J. Biol. Chem. 265:8558 8565. Hydroxylase activity can also be measured using carbon dioxide (¹⁴CO₂ capture assay) in a 96-well microtiter plate format (Zhang et al., 1999, Anal. Biochem. 271:137-142). These assays are readily automated and suitable for high throughput screening of candidate compounds to identify those with hydroxylase inhibitory activity, or to screen for those compounds that decrease the levels of AAH protein in any event.

A screening method used to determine whether a candidate compound inhibits AAH enzymatic activity (as an indirect measure of the AAH protein in the sample) includes the following steps: (a) providing a sample of leukemic cells (which over-produce AAH); (b) providing a polypeptide comprising an EGF-like domain (as substrate for AAH); (c) contacting the sample and the EGF-like polypeptide with the candidate compound for a predetermined period of time; and (d) determining the extent of hydroxylation of the EGF-like polypeptide. A decrease in hydroxylation in the presence of the candidate compound compared to that in the absence of said compound in a control sample of leukemic cells indicates that the compound (1) indirectly inhibits expression of the AAH enzyme, or (2) directly inhibits the enzymatic activity of AAH (i.e., the hydroxylation of EGF-like domains in proteins such as NOTCH, a naturally-occurring substrate of AAH). Anti-tumor agents that directly inhibit AAH activation of NOTCH can also be identified by (a) providing a leukemic cell sample expressing AAH; (b) contacting the cell sample with a candidate compound; and (c) measuring translocation of activated NOTCH to the nucleus of said cell. Translocation is measured by using a reagent such as an antibody which binds to a 110 kDa activation fragment of NOTCH. A decrease in translocation in the presence of the candidate compound compared to that in the absence of the compound in a control sample of leukemic cells indicates that the compound inhibits AAH activation of NOTCH, thereby inhibiting NOTCH-mediated signal transduction and proliferation of AAH-overexpressing tumor cells. See further, Wands et al. US patents, supra.

With any of the above screening assays, the test compound is preferably added to the cell sample for a specified period of time, on the order of minutes or hours, prior to measuring AAH expression. Control leukemic cell samples are incubated without the test compound or with a placebo or vehicle alone.

Determining Responsiveness/Sensitivity to Therapy

In another aspect of the present invention, one can use AAH expression (and, correspondingly, AAH protein levels or enzymatic activity) to determine if an individual subject will respond to a particular drug (or combination of drugs).

In a preferred embodiment, the present invention provides methods, compositions, and kits useful for identifying sensitivity or resistance to a tyrosine kinase inhibitor, more particularly an ABL kinase inhibitor (such as Gleevec®). The ability to accurately predict ab initio whether a patient will be sensitive or resistant to a particular therapy, or whether over a course of therapy a patient is no longer responding to a drug, provides valuable information to the clinician to formulate treatment strategies better tailored to the individual's needs and prognosis.

In certain leukemic cells, such as those of CML and ALL, a reciprocal translocation between human chromosomes 9 and 22 results in an abnormal BCR/ABL fusion gene, known as the Philadelphia chromosome. BCR/ABL-mediated tyrosine phosphorylation appears to promote the transformation of hematopoietic progenitor cells into chronic myeloid and acute lymphocytic leukemias.

The discovery of the BCR/ABL fusion gene prompted the development of the drug, imatinib mesylate (Gleevec®), which is now the first-line drug of choice in the treatment of CML. Gleevec® binds to the BCR/ABL protein and inhibits its enzymatic activity, and thus acts to control the pathology presumed to result from this kinase. However, while Gleevec® has been very successful in controlling CML, a significant number of patients relapse during therapy (Sawyers et al., 2002; Talpaz et al., 2002; Druker et al., 2001). Moreover, there is a small, but significant, population of patients that will not respond to the drug at all. The incidence of relapse and of non-responders is of great concern to clinicians and patients alike, particularly since tyrosine kinase inhibitors (TKIs) like Gleevec® can have serious side effects; thus, ideally, the drug should not be used unless it can provide clinical relief. In other words, the deleterious side effects of Gleevec® in such patients far outweigh the drug benefit, and so it would be extremely advantageous to know whether a patient is a responder or not before initiating or continuing therapy.

There are others in the art who have disclosed methods for determining sensitivity (or conversely, non-responsiveness) to Gleevec® by, for instance, observing arrays of proteins or by directly measuring BCR/ABL. However, the method of the present invention provides a simple (i.e., one biomarker level as opposed to an array of multiple markers) and accurate way to make this assessment, even before treatment has been initiated. In fact, this method is shown to be more reliable than measuring BCR/ABL directly, as described in the Examples.

The present invention now shows that a statistically significant decrease in AAH gene expression directly correlates with a favorable response to Gleevec®, even though the target of Gleevec® is a tyrosine kinase encoded by the BCR/ABL gene. Interestingly, expression levels of BCR/ABL do not correlate with responsiveness to the drug, nor does another cancer biomarker, the Ki67 gene.

For instance, prior to treatment, one can assess an individual's responsiveness to tyrosine kinase inhibition (TKI) therapy by analyzing a sample containing leukemia cells from the individual for diminished expression (or protein or enzymatic activity) levels of AAH after in vitro exposure of the sample to the drug. If after exposure to the tyrosine kinase inhibitor the expression of AAH is not significantly lessened, that is if there is less than an about 30% decrease in AAH expression (and preferably, less than 30% decrease), it is indicative of long-term drug resistance. Conversely, our studies have shown that a decrease of greater than 30% in mRNA levels is suggestive of long-term drug response.

In yet another aspect of the present invention, there is provided a method for monitoring a course of a therapeutic treatment in an individual being treated for leukemia, comprising: a) obtaining a blood sample at a first time point from a patient undergoing said treatment; b) detecting expression of the HAAH gene by assaying for HAAH mRNA or HAAH polypeptide gene product; and c) repeating steps a) and b) at determined time points during the course of treatment, whereby the therapeutic treatment is temporally monitored by detecting any changes in expression of the HAAH gene, and wherein the decreased expression of the HAAH gene is associated with the success of the therapeutic treatment, and increased HAAH gene expression is indicative failure of the treatment. Of course, ever increasing HAAH expression over time in the course of treatment is indicative of acquiring non-responsiveness and reason to change therapeutic modalities.

To assess an individual's expression level of AAH, any conventional method known in the art may be used. By way of example, one may use a quantitative PCR of RNA extracted from the leukemic cell sample to determine AAH expression at the mRNA level (such as TaqMan®, available from Applied Biosystems; Foster City, Calif.). Primers and probes can be obtained or synthesized based on the known sequence of HAAH, and an example of a set of primers and a probe are given in the examples.

Alternatively, one may use immunological methods with labeled antibodies to AAH to detect levels of AAH protein. The methods for analyzing or measuring AAH are conventional and well known to those skilled in the art or may be readily implemented without undue experimentation.

As with all the methods of the present invention, in monitoring treatment or assessing relapse, the levels of AAH expression can be determined by measuring AAH nucleic acid (such as by reverse transcriptase PCR to measure HAAH mRNA) or measuring the AAH polypeptide itself (such as by an immunological test using anti-AAH antibodies, which are known and available), it being understood that one or the other, or even both such molecular entities can be monitored, as long as the overall monitoring is done consistently and the treatment being assessed is not an anti-AAH antibody if the assay used is immunological for the polypeptide. Of course, increased AAH expression at any time during the course of treatment is indicative of non-responsiveness over time and reason to change therapeutic modalities.

The assay format described below may be used to screen potential anti-leukemia agents or to generate temporal data used for long-term therapeutic effectiveness or prognosis of the disease. While the assay exemplified below is nucleic acid based, a protein expression assay is also contemplated as applicable to make the determinations as well. For example, such a protein expression type of assay is carried out by contacting a sample containing lymphocytes from the mammal (in practical terms, a human) with an antibody that specifically binds to an HAAH polypeptide under conditions sufficient to form an antigen-antibody complex and detecting the antigen-antibody complex, and quantitating the amount of complex to determine the level of HAAH in the sample. Increased levels of the enzyme have been correlated with increased gene expression. Thus, such an assay can be used to diagnose, measure efficacy of a drug candidate, or chart the prognosis or the effectiveness of a course of therapy over time. An increasing level of HAAH over time indicates a progressive worsening of the disease, and therefore, an adverse prognosis, or lack of continued effectiveness of the therapy.

The assay used below is of a quantitative RT-PCR format, which measures mRNA, and is well known to those in the art. The sequences of the HAAH polypeptide and the HAAH cDNA are known from U.S. Pat. Nos. 6,797,696; 6,783,758; 6,812,206; 6,815,415; 6,835,370; and 7,094,556, all of which are specifically incorporated in their entireties herein by reference, and the knowledge of which will allow one of ordinary skill to readily determine and obtain the assay reagents for this and other assay types. Thus, the present invention is not limited to any particular assay reagents or format, as long as HAAH gene/protein expression is the measurable endpoint.

Further, to determine loss of sensitivity to a therapeutic agent during the course of therapy in accordance with the methods herein, a patient's blood sample can be first weaned off the therapeutic agent by culturing the cells for 24-48 hours, with one or more media changes, prior to assessing sensitivity of the cells with the therapeutic agent. Also, an alternative to culturing leukocytes, the assay may be conducted immediately on non-cultured whole blood samples.

There is no particular limitation on the test sample that can be used in the methods of the present invention, only that it contains white blood cells, for example peripheral blood, lymph node tissues or fluid, or bone marrow tissue or fluid.

The invention is further illustrated by the following examples, which are not intended to limit the scope thereof.

### EXAMPLES

### Example 1

### RT-PCR TaqMan® Assay for Catalytic Form of HAAH

This assay procedure allows the quantitative comparison of test RNA to a standard curve using single tube TaqMan® reactions.

Materials:
MilliQ H2O
1.5 ml microtubes
5 ml tubes
Microtube racks
Pipettes (P-20, P-200, P-1000)
Pipette tips
TaqMan EZ RT-PCR Core Reagents (Perkin Elmer #N808-0236)
Primers for target HAAH (or ASPH) mRNA (Commonwealth BI)

| | |
|---|---|
| ASPHBFwd: TGTGCCAACGAGACCAAGAC | [SEQ ID NO:1] |
| ASPHBRev: CGTGCTCAAAGGAGTCATCAAA | [SEQ ID NO:2] |

Hybridization Probe (CBI)

[SEQ ID NO:3]

ASPHBHyb: 6FAM-AGGCAAGGTGCTCAT-MGBNFQ

Optical 96 well plates
Adhesive seal

General Procedure:

Setting up EZ RT-PCR for Taqman at 5 mM MnOAc:

I. ASPH/B Primer Sets and Probe:
Working Solution 9 μM Forward Primer—9 μM=70.31 μl (128 μM)+929.69 μl H2O
Working Solution 9 μM Reverse Primer—9 μM=68.7 μl (131 μM)+931.3 μl H2O
Working Solution 2 μM Probe—2 μM=20 μl (100 μM)+980 μl H2O
Positive Control: ASPH/B (76 ng/μl)

A clone that is known to contain the primer and probe sequences, named ASPH-B, is used to generate a standard curve.

$$Dilute positive control - 10 ng \text{/} µl = 13.2 µl ( 76 ng \text{/} µl ) + 86.6 µl H 2 O$$

$$1 ng \text{/} µl = 10 µl ( 10 ng \text{/} µl ) + 90 µl H 2 O$$

Prior to using, serial dilutions are made by adding H2O to labeled tubes, then add 3 μl of the previous dilution and vortex.

106=3 μl (1 ng/μl)+297 μl H2O

104=3 μl (106)+297 μl H2O

102=3 μl (104)+27 μl H2O

101=3 μl (103)+27 μl H2O

100=3 μl (102)+27 μl H2O
Use 1 μl standard
Negative Control Use 1 μl H2O

II. Master Mix:
Per sample: 5.5 μl H2O
   10.0 μl 5×EZ Buffer
   10.0 μl 25 mM MnOAc
   1.5 μl 10 mM dATP
   1.5 μl 10 mM dCTP
   1.5 μl 10 mM dGTP
   1.5 μl 20 mM dUTP
   5.0 μl 9 μM F
   5.0 μl 9 μM R
   5.0 μl 2 μM H
   2.0 μl rTth
   0.5 μl UNG

Aliquot 49 μl master mix into all of the wells, then, after vortexing sample, add 1 μl of sample (i.e., positive control, negative control, or unknown) into each well of an optical grade 96 well plate. Seal wells. Vortex plate, tap bubbles out.

Set up plate as described in Perkin Elmer sequence detection system manual.

Place optical plate in PE 7700 and run using the following conditions: 50 degrees C.-2 min, 60 degrees C.-30 min, 95 degrees C.-5 min, 94 degrees C.-20 sec, 62 degrees C.-1 min, 40 cycles.

Analyze as described in Perkin Elmer sequence detection system manual. A standard amplification curve of ASPH-B was obtained previously, to which unknown samples are compared to obtain the number of copies of HAAH mRNA in the sample.

Patient leukocytes were isolated from patient blood samples and grown in culture in the presence or absence of imatinib mesylate. After overnight incubation, qRT-PCR was used to determine human aspartyl (asparaginyl) β-hydroxylase (HAAH) mRNA expression in both treated and untreated cells. Percentage decrease in mRNA expression level is determined by comparing expression levels in treated vs. non-treated leukocytes from each individual patient.

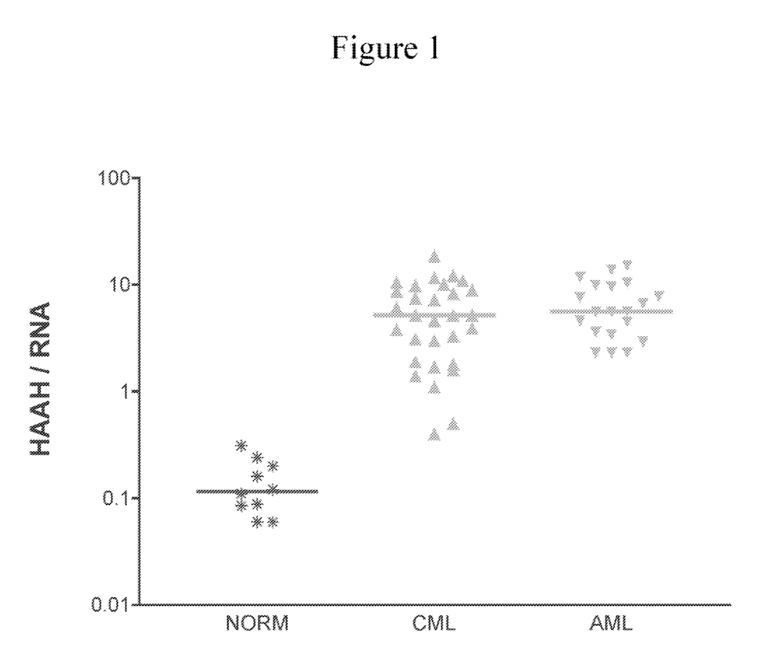

After overnight treatment of patient leukocytes with imatinib mesylate, HAAH mRNA is measured in the test sample and the control untreated sample, and the percentage decrease in expression of HAAH mRNA of test vs. control is determined. The range indicating sensitivity to imatinib is determined as a >30% decrease. This percentage change is consistent with a prediction of sensitivity to imatinib mesylate, based on numerous studies done by the present inventors. Such a change in the expression levels of HAAH mRNA after an overnight in vitro treatment of leukocytes with imatinib mesylate have been demonstrated to be an indicator of patient response to the drug. Studies have shown that a decrease of greater than 30% in mRNA levels is suggestive of long-term drug response. The above methods were used to generate the data in the graphs shown in FIGS. 1-4.

In particular, leukocytes from 39 patients were isolated from fresh whole blood prior to the initiation of therapy and cultured for 24 hours in the presence or absence of 1 μM imatinib. HAAH and BCR/ABL transcript levels were determined by real-time quantitative polymerase chain reaction (qRT-PCR) analysis. Patients were treated with imatinib and their response status was assessed vis-à-vis complete molecular remission (CMR) by qRT-PCR of the BCR-ABL fusion gene.

Prior to treatment, all patient samples had increased expression of the HAAH transcript (˜5-fold). The leukocytes of 27 patients displayed a 30-75% decrease in HAAH expression after culture in the presence of imatinib. All of the corresponding patients achieved CMR after drug therapy. The leukocytes of the 12 other patients displayed less than a 25% reduction in HAAH transcript levels and these patients proved to be non-responders to drug treatment. Transcript levels of either the BCR-ABL gene itself or a control gene, Ki67, did not correlate with drug response.

Decreased levels of expression of the HAAH transcript after a 24 hour in vitro exposure of primary leukocytes to imatinib is a simple and sensitive assay for the determination of likely response to imatinib prior to the initiation of treatment. Based on these results, this assay also represents a quick and simple approach to high throughput screening for new drug candidates against CML (and other Philadelphia chromosome-positive disease states).

In FIGS. 1-4, KO indicates untreated cells (100% of the original expression); BCR/ABL+ indicates positive control (K562 cell lines (CML-derived)); BCR/ABL− indicates negative control (healthy donors, AML patients); CML indicates patients before Gleevec® therapy; ODP indicates Responders (responded well to Gleevec®); and NEODP indicates Non-Responders (Gleevec® therapy failed).

FIG. 1 shows there is an 80-fold higher level of HAAH gene expression in peripheral blood cells of leukemia patients vs. healthy donors. Thus, HAAH can serve as a biomarker for leukemia diagnosis, as well as for assessing drug efficacy.

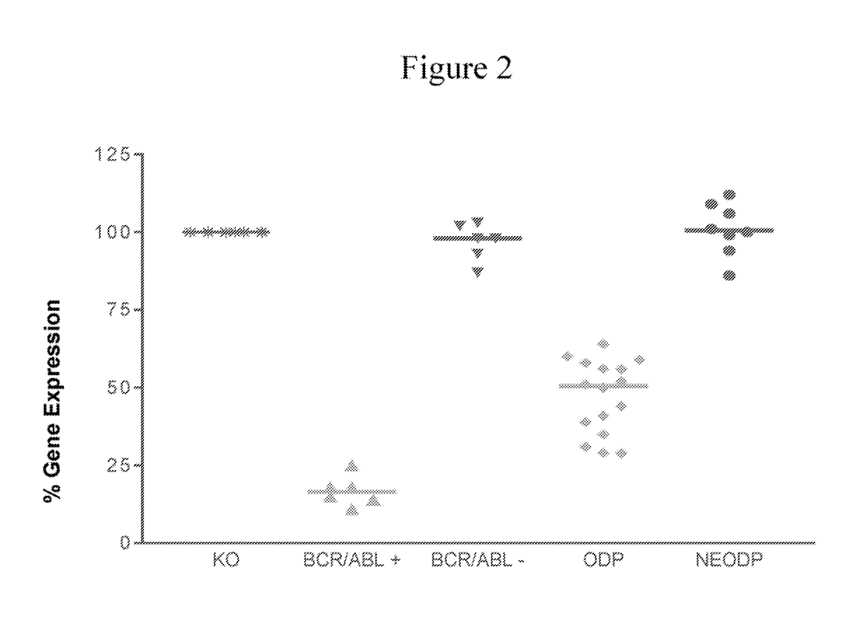

FIG. 2 shows that changes in HAAH gene expression level upon in vitro treatment of leukemic PBLs with Gleevec® correlate with patient response to the drug, with decreased levels being indicative of a nonresponder. Thus, HAAH detection can be used as a tool to predict Gleevec® resistant patients even prior to treatment.

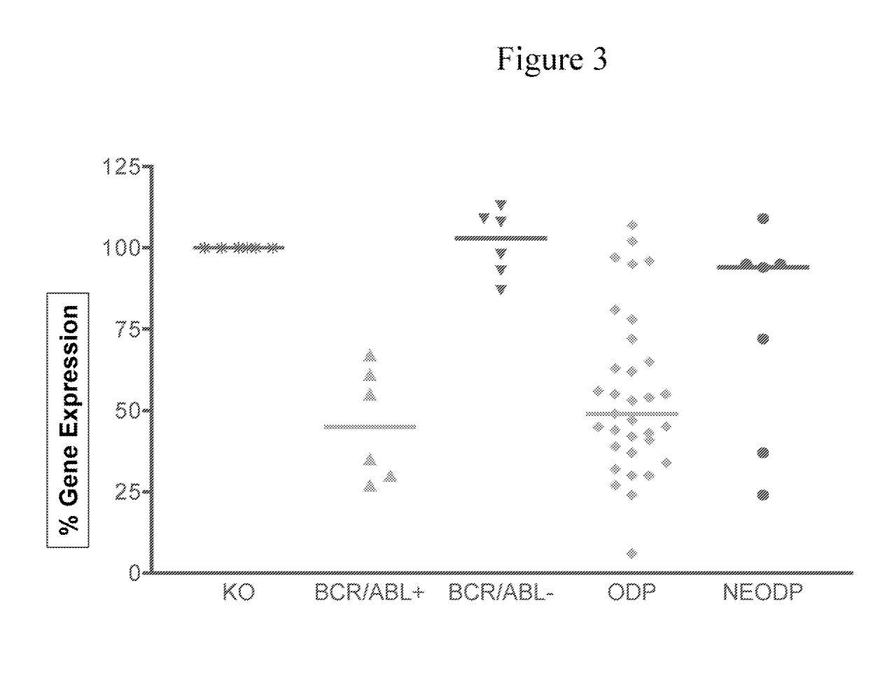

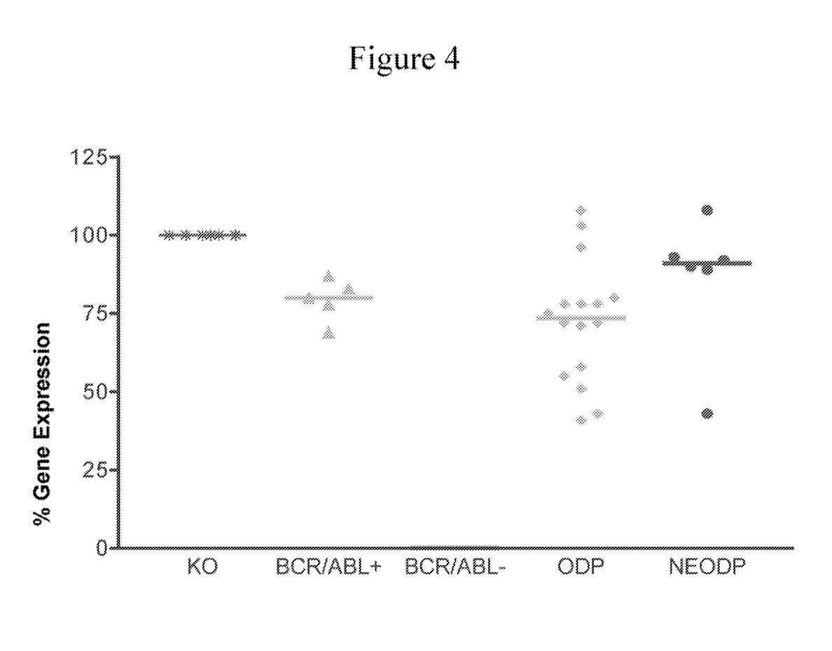

FIGS. 3 and 4 show that Ki67 and BCR-ABL gene expression, respectively, are not predictive of responsiveness to Gleevec®.

### Example 2

Acute myelogenous leukemia (AML) is characterized by the rapid proliferation of immature leukocytes (blasts) in patient peripheral blood (PB). Initial diagnosis of AML relies upon blood cell counts and detection of cytomorphological changes in leukocytes found in the PB and bone marrow (BM). Treatment of AML begins with induction chemotherapy resulting in complete remission (CR) in 60-80% of all patients. CR is defined as the absence of leukemic cells in both the PB and BM as detected by cytomorphological assessment. Due to the lack of sensitivity of these methods, small numbers of diseased cells may persist resulting in disease relapse. Thus, in most cases multiple rounds of chemotherapy may be recommended. A specific molecular marker for minimum residual disease (MRD) would be of great benefit in determining prognosis, optimal post-remission therapy, effectiveness of therapy, and as an early indicator of disease relapse.

Human aspartyl (asparaginyl) β-hydroxylase is a highly specific biomarker for metastatic cancer. Increased HAAH expression has previously been detected at the protein and mRNA levels specifically in tumor cells. Overexpression of HAAH results in its translocation to the cellular surface where it is a potential target for antibody-based cancer therapy. The primary object of this example was to investigate whether HAAH gene expression levels are a marker for MRD in AML.

Expression levels of HAAH were determined by real-time quantitative polymerase chain reaction (qRT-PCR) analysis of leukocytes isolated from fresh whole blood at diagnosis of AML and compared to healthy donors. Expression levels in treated patients who attained CR were also determined.

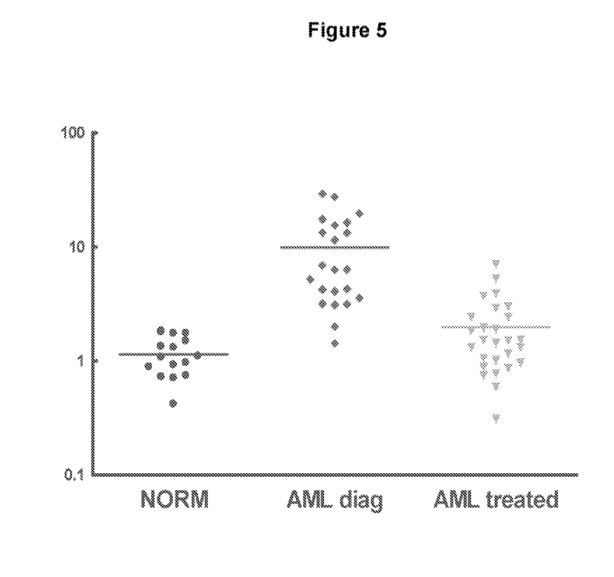

At diagnosis, patients (n=22) displayed increased expression of the HAAH transcript (˜8.6-fold, p<0.0002) when compared to healthy donors (n=15). Increased levels of HAAH expression were detected in multiple AML subtypes. On average, HAAH expression decreased in treated patients (n=27) to essentially normal levels. See FIG. 5.

The results show that HAAH expression represents a molecular marker for AML that is broadly applicable over multiple disease subtypes and is of assistance in monitoring remission or relapse. Moreover, HAAH expression is easily determined from PB leukocytes and would be useful in replacing bone marrow biopsy as a monitoring tool. Thus, determination of HAAH expression levels enhances the diagnosis and monitoring of AML.

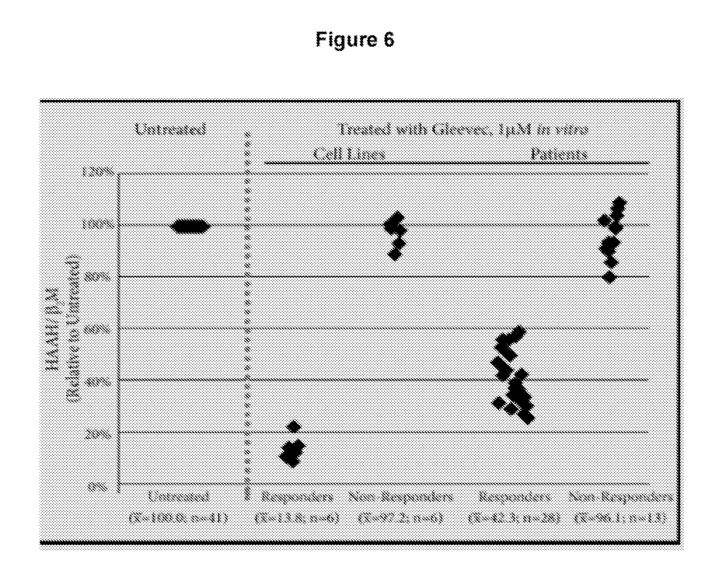

FIG. 6 shows that HAAH expression levels are diagnostic for myelogenous leukemias, and that HAAH expression levels correlate with induction of remission in AML. These results show that qRT-PCR for HAAH is sensitive and can detect increased gene expression in even a few diseased cells, and that qRT-PCR for HAAH has utility in the detection of MRD and AML relapse

We claim:

1. A method for determining whether a potential therapeutic agent is useful in the treatment of leukemia, comprising: contacting the agent with a quantity of known leukemic cells in vitro, and detecting the level of expression of human aspartyl (asparaginyl) β-hydroxylase (HAAH), whereby a decrease in the expression of HAAH, relative to the level of HAAH expression in a control containing the same quantity of leukemic cells in the absence of the agent, indicates a positive response to the agent.

2. The method of claim 1, wherein the level of expression of HAAH is determined by measuring HAAH mRNA.

3. The method of claim 3, wherein the mRNA level is measured using an RT-PCR assay.

4. The method of claim 1, wherein the level of expression of HAAH is determined by measuring the level of HAAH polypeptide.

5. The method of claim 4, wherein the HAAH polypeptide level is determined by using an immunological assay.

6. The method of claim 1, wherein the potential therapeutic agent is a small molecule, an antibody, or an antisense polynucleotide.

7. The method of claim 1, wherein the leukemia is chronic myelogenous leukemia, acute myelogenous leukemia or Philadelphia chromosome positive acute lymphocytic leukemia.

8. A method of monitoring clinical success of a therapeutic treatment for leukemia, comprising: a) obtaining a blood sample at a first time point from a patient undergoing said treatment; b) detecting expression of the HAAH gene by assaying for HAAH mRNA or HAAH polypeptide levels; and c) repeating steps a) and b) at determined time points during the course of treatment, whereby the therapeutic treatment is temporally monitored by detecting any changes in expression of the HAAH gene, and
wherein the decreased expression of the HAAH gene over time is associated with the success of the therapeutic treatment, and increased HAAH gene expression over time is indicative failure of the treatment.

9. The method of claim 8, wherein the level of expression of HAAH is determined by measuring HAAH mRNA.

10. The method of claim 9, wherein the mRNA level is measured using an RT-PCR assay.

11. The method of claim 8, wherein the level of expression of HAAH is determined by measuring the level of HAAH polypeptide.

12. The method of claim 11, wherein the HAAH polypeptide level is determined with an immunological assay.

13. The method of claim 8, wherein the leukemia is chronic myelogenous leukemia, acute myelogenous leukemia or Philadelphia chromosome positive acute lymphocytic leukemia.

14. A method for identifying whether a patient's leukemia condition is sensitive or resistant to a particular therapeutic agent prior to treatment therewith, comprising: a) obtaining a blood sample from the patient; b) contacting a portion of the sample with the agent in vitro to serve as a test sample, and reserving a portion of the sample to serve as a control to which the agent is absent; c) detecting expression level of the HAAH gene by assaying for HAAH mRNA or HAAH polypeptide in the test and control samples,
whereby a decreased level of expression of HAAH, relative to the level of HAAH expression in a control containing the leukemic cells in the absence of the agent, indicates the patient's leukemia is sensitive to the agent.

15. The method of claim 14, wherein the level of expression of HAAH is determined by measuring HAAH mRNA.

16. The method of claim 15, wherein the mRNA level is measured using an RT-PCR assay.

17. The method of claim 14, wherein the level of expression of HAAH is determined by measuring the level of HAAH polypeptide.

18. The method of claim 14, wherein the HAAH polypeptide level is determined with an immunological assay.

19. The method of claim 14, wherein the leukemia is chronic myelogenous leukemia, acute myelogenous leukemia or Philadelphia chromosome positive acute lymphocytic leukemia.

20. The method of claim 14, wherein the therapeutic agent is a small molecule, an antibody, or an antisense polynucleotide.

21. The method of claim 20, wherein the therapeutic agent is a tyrosine kinase inhibitor.

22. The method of claim 20, wherein the therapeutic agent is imatinib mesylate.

23. A method for identifying whether a patient has leukemia, comprising:
obtaining a blood sample from said patient; and b) detecting an expression level of the HAAH gene by assaying for HAAH mRNA or HAAH polypeptide, and comparing said level to a normal non-leukemia control, whereby an increase in the expression of HAAH, relative to the level of HAAH expression in the control, indicates a positive result for leukemia.

24. The method of claim 23, wherein the leukemia is chronic myelogenous leukemia, acute myelogenous leukemia or Philadelphia chromosome positive acute lymphocytic leukemia.

25. A kit comprising a set of amplification primers and a probe for HAAH, and instructions for using the same to measure HAAH in a test sample in order to diagnose leukemia in the sample or to determine if the subject will respond or not to a certain treatment or therapeutic agent.

26. The kit of claim 25, wherein said primers are SEQ ID NO:1 and SEQ ID NO:2, and the probe is SEQ ID NO:3.
